(19) United States
(12) Patent Application Publication
(10) Pub. No.: US 2013/0226123 A1
(43) Pub. Date: Aug. 29, 2013

(54) ABSORBENT ARTICLE AND SANITARY NAPKIN

(71) Applicants: Jun Kudo, Kagawa (JP); Hideyuki Kinoshita, Kagawa (JP); Yuji Takahashi, Kagawa (JP); Mari Minami, Kagawa (JP)

(72) Inventors: Jun Kudo, Kagawa (JP); Hideyuki Kinoshita, Kagawa (JP); Yuji Takahashi, Kagawa (JP); Mari Minami, Kagawa (JP)

(21) Appl. No.: 13/819,719

(22) PCT Filed: Aug. 31, 2011
(86) PCT No.: PCT/JP2011/004889

§ 371 Date: May 9, 2013

(30) Foreign Application Priority Data
Aug. 31, 2010 (JP) 2010-195078

(51) Int. Cl. A61F 13/538

(52) U.S. Cl. CPC A61F 13/538
USPC 604/380; 604/378

(57) ABSTRACT

An absorbent article has an upper layer unit which extends in a longitudinal direction of the absorbent article is provided on a skin contact surface side of the main body unit. The upper layer unit has an upper layer topsheet which is independent of the topsheet. A dimension of the upper unit in a widthwise direction of the absorbent article is configured to be smaller than a dimension of the main body unit in the widthwise direction. A central region including a center of the widthwise direction of the main absorber that is opposite to the upper layer unit is provided, and in at least a part of the central region, a low rigidity region of which flexural rigidity is lower than that of the central region is formed.

### TECHNICAL FIELD

The present invention relates to an absorbent article, and in particular, to a sanitary napkin.

### BACKGROUND ART

Conventionally, it is known that there is provided an absorbent article having a main body unit which includes a topsheet, a backsheet, and a main absorber, in which an upper layer unit extending in a longitudinal direction is provided at a skin contact surface side of such a main body unit (see Patent Document 1, for example).

In such an absorbent article, a dimension of an upper layer unit in a widthwise direction are adapted so as to be smaller than that of the main body unit in the widthwise direction; and at the main body unit, a region in which the main absorber is not formed is formed in a region which is opposite to the upper layer unit.

### CITATION LIST

### Patent Literature

PTL 1: Japanese Patent Application Laid-open No. 2006-115996

### SUMMARY OF INVENTION

### Technical Problem

In such an absorbent article, in a state in which the main absorber and the upper layer absorber are covered with the same topsheet, since the main absorber and the topsheet are bonded with each other, the main absorber is hardly deformed in a widthwise direction, with a recessed unit being a start point, due to an influence of such a topsheet; and therefore a fitting property for an wearer is lost.

In addition, in such an absorbent article, since the upper layer absorber is established in a state in which a degree of freedom is restrained between the main absorber and the topsheet, when the entire absorbent article is curved in a longitudinal direction, the upper layer absorber cannot reduce a distance merely by manually handling it; the main absorber is hardly deformed in the widthwise direction, with the recessed unit being the start point; and therefore, the fitting property for a wearer is lost.

Accordingly, the present invention has been made in view of the situation described above, and it is an object of one or more embodiments of the present invention to provide an absorbent article or a sanitary napkin which is capable of facilitating deformation in a widthwise direction of a main absorber and improving the fitting property for a wearer.

### Solution to Problem

The first feature of the present invention is summarized in that an absorbent article having: a main body unit including a topsheet; a backsheet; and a main absorber unit which is disposed between the topsheet and the backsheet, wherein: an upper layer unit which extends in a longitudinal direction of the absorbent article is provided on a skin contact surface side of the main body unit; the upper layer unit has an upper layer sheet which is independent of the topsheet; a dimension of the upper layer unit in a widthwise direction of the absorbent article is configured to be smaller than a dimension of the main body unit in the widthwise direction; a central region including a center of the widthwise direction of the main absorber that is opposite to the upper layer unit is provided; and in at least a part of the central region, a low rigidity region of which flexural rigidity is lower than that of the central region is formed.

The second feature of the present invention is summarized in that a sanitary napkin including a main body unit having a topsheet, a backsheet, and a main absorber which is disposed between the topsheet and the backsheet, wherein: the main body unit has a central wing unit which extends outwardly in a widthwise direction from opposed longitudinally extending side edges of the sanitary napkin and a rearward wing unit which is provided rearward of the central wing unit in a longitudinal direction of the sanitary napkin and which extends outwardly in the widthwise direction from opposed longitudinally extending side edges of the sanitary napkin; an upper layer unit extending in the longitudinal direction of the sanitary napkin is provided at a skin contact surface side of the main body unit; the upper layer unit has an upper layer topsheet which is independent of the topsheet and an upper layer absorber which is independent of the main absorber; a dimension of the upper layer unit in the widthwise direction of the sanitary napkin is configured to be smaller than a dimension of the main body unit in the widthwise direction; a central region is provided in the main absorber, which includes a widthwise center of the main absorber that is opposite to the upper layer unit;

in the central region, in a region at least a part of which is opposite to a region in which the upper layer absorber is disposed in the upper layer unit, a low rigidity region is formed which is arranged to have a lower flexural rigidity than that of the central region; the low rigidity region is formed rearward in the longitudinal direction of a region in which a dimension of the main body unit in the widthwise direction is the smallest;

the rearward wing unit is provided outside of the low rigidity region in the widthwise direction; the main body unit and the upper layer unit are not bonded with each other in a region which is opposite to the low rigidity region; a dimension of the low rigidity region in the widthwise direction is configured to be smaller than the dimension of the upper layer absorber in the widthwise direction; at least a first compression groove and a second compression groove which are separated from each other are provided on the skin contact surface side of the main body unit; the first compression groove extends in the longitudinal direction outside of the low rigidity region in the widthwise direction; the second compression groove extends in the longitudinal direction outside of the low rigidity region in the widthwise direction and extends in the widthwise direction rearward in the longitudinal direction of a region which is opposite to the low rigidity region; and a basis weight of the main absorber in the low rigidity region is configured to be lower than a basis weight of the main absorber in a peripheral region of the low rigidity region.

In the above feature of the present invention, the upper layer unit may extend over the entire length of the absorbent article and the upper layer absorber may extend for a partial length of the upper layer unit only and is provided in a rearward region only such that it is spaced from a front edge of the absorbent article by a greater distance that it is spaced from a rear edge of the absorbent article. Further, the upper layer absorber may overlap the low rigidity region.

### Advantageous Effects of Invention

As has been described above, according to one or more embodiments of the present invention, there can be provided an absorbent article and a sanitary napkin which are capable of facilitating deformation in a widthwise direction of a main absorber and improving the fitting property for a wearer.

### BRIEF DESCRIPTION OF DRAWINGS

FIG. 1 is a plan view seen from a skin contact surface side of an absorbent article according to a first embodiment of the present invention.

FIG. 2 is a sectional view taken along the line A-A′ and a sectional view taken along the line B-B′, of the absorbent article according to the first embodiment of the present invention.

FIG. 3 is a view for explaining a method of manufacturing the absorbent article according to the first embodiment of the present invention.

FIG. 4 is a plan view seen from a skin contact surface side of an absorbent article according to Exemplary Modification 1 of the present invention.

FIG. 5 is a sectional view taken along the line C-C′ of the absorbent article according to Exemplary Modification 1 of the present invention.

### DESCRIPTION OF EMBODIMENTS

### First Embodiment of the Present Invention

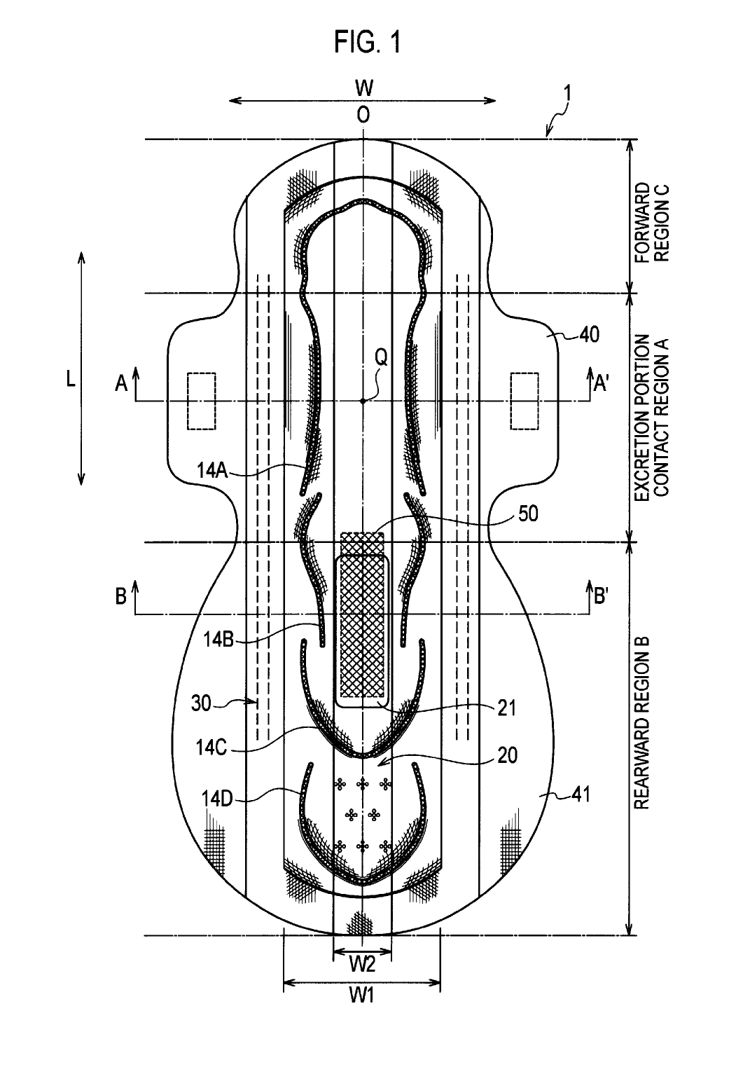

An absorbent article 1 according to a first embodiment of the present invention will be described with reference to FIG. 1 to FIG. 3. For example, the absorbent article 1 according to the embodiment may be a diaper, an incontinence pad, a sanitary napkin or the like. Hereinafter, the absorbent article 1 according to the embodiment will be described by way of example as a sanitary napkin. The present embodiment is not, however, limited to a sanitary napkin.

As shown in FIG. 1, the absorbent article 1 is comprised of an excretion portion contact region A, a rearward region B, and a forward region C.

The rearward region B is disposed at a rear side in a longitudinal direction L of the absorbent article 1 with respect to the excretion portion contact region A, and the forward region C is disposed at a fore side in the longitudinal direction of the absorbent article 1 with respect to the excretion portion contact region A.

The excretion portion contact region A is a region of the absorbent article 1 which comes into contact with a wearer's vagina, and for example, is a region which is disposed between two leg-line apertures of the wearer's underwear when the absorbent article 1 is worn on the wearer's underwear.

In addition, the absorbent article 1 has: one pair of central wing units 40 which are formed so as to extend to both of the outsides of a widthwise direction W, in the excretion portion contact region A; and a rearward wing unit 41 which is formed so as to extend to the outside in the widthwise direction W, in the rearward region B.

Here, when wearing the absorbent article 1 on the underwear, the wearer winds the central wing unit 40 around the underwear and then fixes the absorbent article 1 to the underwear. In addition, when wearing the absorbent article 1 on the underwear, the wearer fixes the rearward wing unit 41 in a state in which it opens at the rear side of the wearer's body on which the underwear is worn.

In addition, in the absorbent article 1, it may be that a crossing point Q, which is a crossing point between a line O in the longitudinal direction L, which passes through a center of the widthwise direction W (i.e. the longitudinal centerline O), and a line (A-A′) in the widthwise direction W, which passes through a center of the central wing unit 40 in the longitudinal direction L is defined as a “center of the excretion portion contact region A in the longitudinal direction L”; wherein a region between a site which is preferably distant by 40 mm rearward in the longitudinal direction L from the crossing point Q and a rear end part in the longitudinal direction L is defined as the “rear region B”; and a region between a site which is preferably distant by 40 mm forward in the longitudinal direction L from the crossing point Q and a forward end part in the longitudinal direction L is defined as the “forward region C”.

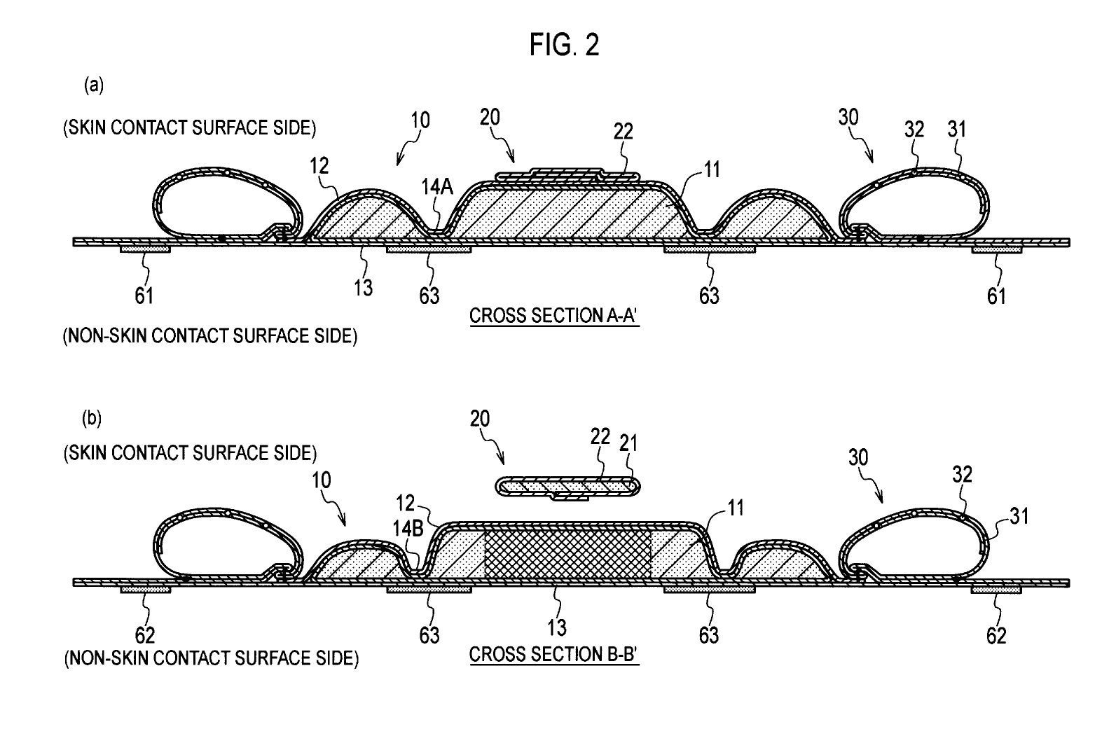

As shown in FIG. 2(a) and FIG. 2(b), the absorbent article 1 of the embodiment has a main body unit 10 and an upper layer unit 20.

Here, the main body unit 10 has: a topsheet 12; a backsheet 13; and a main absorber 11 which is disposed between the topsheet 12 and the backsheet 13.

The topsheet 12 is a liquid permeable sheet which is provided at the skin contact surface side of the absorbent article 1 in the main body unit 10, and the backsheet 13 is a liquid impermeable sheet which is provided at the non-skin contact surface side of the absorbent article 1 in the main body unit 10.

For example, the topsheet 12 is an air-through unwoven cloth of which basis weight is 25 g/m², and is comprised of a texture of which a core of 2.2 dtex is PET and a cladding is PE. A bulk recovery treatment (a treatment of leaving at a temperature of 90 degrees for 10 minutes) is not applied to such topsheet 12. The density of such a topsheet 12 is 0.055 g/cm³.

In addition, the backsheet 13 is comprised of a polyethylene film of 20 g/m², for example.

The main absorber 11 is configured to absorb bodily fluid such as urine or menstrual blood, and is disposed over the excretion portion contact region A, the rearward region B, and the forward region C in the absorbent article 1.

For example, as the main absorber 11, there may be used a plurality of laminations of tissue of the order of 15 g/m² or spun lace nonwoven cloth including rayon or the like, or alternatively, an air-laid pulp sheet or the like including an absorptive polymer or a functional material. A material for the main absorber 11 is not limited in particular as long as it is a material which is capable of absorbing bodily fluid.

As shown in FIG. 1, the main absorber 11 and the topsheet 12 are bonded with each other by means of a plurality of compression grooves 14A, 14B, 14C, and 14D.

In such an absorbent article 1, a leakage preventing wall 30 may be provided at each side in the widthwise direction W of the main body unit 10. Such a leakage preventing wall 30 is comprised of: a leakage proof sheet 31 which is folded on a hollow; and an elastic member 32 which is disposed in an extendable state in the leakage proof sheet 31.

In addition, it may be that: the leakage proof sheet 31 is comprised of a spun bond nonwoven cloth of 10 to 30 g/m²; and the elastic member 32 may be comprised of a yarn-like natural runner or a synthetic rubber.

Further, the upper layer unit 20 is provided so as to extend to the skin contact surface side of the main body unit 10 in the longitudinal direction L. That is, this unit is disposed over a whole length of the absorbent article 1. Here, a dimension W2 of the upper layer unit 20 in the widthwise direction W is configured to be smaller than a dimension W1 of the main body unit 10 in the widthwise direction W.

Specifically, the upper layer unit 20 has: an upper layer topsheet 22 which is independent of the topsheet 12; and an upper layer absorber 21 which is independent of the main absorber 11.

For example, the upper layer topsheet 22 is an air-through unwoven cloth of which basis weight is 20 g/m², and is composed of a texture of which a core of 2.2 dtex is PET and a cladding is PE. A bulk recovery treatment is applied to such a topsheet 12. The density of such a topsheet 12 is 0.025 g/cm³,

For example, as the upper layer absorber 21, there may be used a plurality of laminations of tissue of the order of 15 g/m² or a spun lace nonwoven cloth including rayon or the like, or alternatively, an air-laid pulp sheet or the like including a absorptive polymer or a functional material. A material for the upper layer absorber 21 is not limited in particular as long as it is a material which is capable of absorbing a bodily fluid.

Here, as shown in FIG. 2(a) and FIG. 2(b), the upper layer topsheet 22 is folded into three sections, and regions of the upper layer topsheet 22 that has been folded into three sections are bonded with each other by means of a hot melt adhesive agent (HMA).

In addition, as shown in FIG. 2(b), in the upper layer unit 20 that is disposed in the rearward region B, the upper layer absorber 21 is disposed inside of the upper layer topsheet 22 that has been folded into three sections.

That is, as shown in FIG. 2(a), in the upper layer unit 20 that is disposed in the excretion portion contact region A (and the forward region C), the upper layer absorber 21 is not disposed inside of the upper layer topsheet 22 that has been folded into three sections.

The absorbent article 1 is preferably configured to be packed after it has been folded into four sections. Here, if three folding lines which are formed at the time of packing are a first folding line, a second folding line, and a third folding line, each of which extends in the transverse direction and are provided in sequential order from the rear of the absorbent article 1 and spaced from one another in the longitudinal direction L of the absorbent article 1, such an upper layer absorber 21 is preferably configured to be disposed between the first folding line and the second folding line.

In addition, in the upper layer unit 20, a spacing region (a slide unit) is provided between a region in which the upper layer absorber 21 is not disposed and a region in which the upper layer absorber 21 is disposed.

In such a spacing region, the main body unit 10 and the upper layer unit 20 are not bonded with each other. That is, as shown in FIG. 1 and FIG. 2(b), the upper layer unit 20 is not bonded with the main body unit 10 in at least a part of a region which is opposite to (overlaps) a low rigidity region 50.

According to such a structure, when the absorbent article 1 is curved in the longitudinal direction L, while an arc of a curved unit at the skin contact surface side is smaller in comparison with an arc of a curved unit at the contact face side of a cloth or the like, the upper layer unit 20 is not bonded with the main body unit 10, whereby the upper layer unit 20 readily slides forward or backward, winkles hardly occurs at the upper layer unit; and therefore, the upper layer unit 20 readily keeps track of and captures a bodily fluid that has flowed.

On the other hand, as shown in FIG. 1 and FIG. 2(a), in a region other than such a spacing region, the main body unit 10 and the upper layer unit 20 are bonded with each other by means of a compression unit (an emboss) or a hot melt adhesive agent (RMA) or the like.

In such a case, in a case where the absorbent article 1 is worn on an underwear and then is curved in the longitudinal direction L, such a spacing region (a slide unit) is configured to move forward or rearward in the longitudinal direction L.

In addition, in the upper layer unit 20, the abovementioned spacing region may not be provided. In such a case, the upper layer potion 20 and the main body unit 10 are bonded with each other over the whole length of the product in the longitudinal direction L of the absorbent article 1.

A central region of the main absorber, which includes a center O in the widthwise direction W of the main absorber 11 (i.e. a central region of the main absorber which spans a longitudinal centerline 0 of the main absorber), and which is opposite to (overlaps) the upper layer unit 20 is provided, and in at least a part of such a central area, a low rigidity region 50 which is configured to have a flexural rigidity that is lower than that of the remainder of the central region is formed. For example, the Gurley method (Method A) defined as a testing method of bending repulsion in JISL1096 (Testing methods for woven and knitted fabrics, updated version of 1994) can be employed to measure rigidity values of the low rigidity region 50 and high rigidity region (between the compression grooves 14B and 14C).

More specifically, the low rigidity region 50 is formed in a region of the main absorber 11, at least a part of which is opposite to a region in which the upper layer absorber 21 is disposed in the upper layer unit 20.

According to such a structure, the low rigidity region 50 in the main absorber 11 is deformed so as to be taken along a concave shape of a wearer's body (a concaveness of the wearer's hip) by means of a wearing pressure of underwear, and accordingly, the upper layer unit 20 can also be flexibly deformed so as to be taken along the concave shape of the wearer's body; and therefore, the upper layer unit 20 readily comes into contact with a bodily fluid that flows while a gap in the wearer's body is filled with the fluid. As a result, the upper layer absorber 21 readily absorbs such a bodily fluid immediately.

A dimension of the low rigidity region 50 in the widthwise direction W is configured to be smaller than that of the upper layer absorber 21 in the widthwise direction W. Incidentally, the low rigidity region 50 may be formed rearward of the longitudinal direction L more than a region in which a dimension of the main body unit 11 in the widthwise direction W is the smallest.

Here, since the low rigidity region 50 serves as a deformation start point in the widthwise direction W of the absorbent article 1, if the dimension of the low rigidity region 50 in the widthwise direction W is greater than that of the upper layer absorber 21 in the widthwise direction W, there is a possibility that the absorbent article 1 is deformed outside of the widthwise direction W more than at the upper layer absorber 21, and there is a possibility that a desired function cannot be attained.

A basis weight of the main absorber 11 in the low rigidity region 50 may be configured to be lower than that of the main absorber 11 in a peripheral region of the low rigidity region 50. In addition, such a low rigidity region 50 may be formed by providing a slit at the main absorber 11.

According to such a structure, the absorbent article 1 can be manufactured at low equipment costs by reducing the basis weight of the main absorber 11 in the low rigidity region 50.

The low rigidity region 50 is formed rearward of the longitudinal direction L more than at a central wing unit 40. According to such a structure, the low rigidity region 50 is disposed rearward of the absorbent article 1, whereby the low rigidity region 50 can be fitted to concaveness of the wearer's hip which is the deepest among all of the concave parts of the wearer's body.

In addition, a rearward wing unit 41 is preferably provided outside of the low rigidity region 50 in the widthwise direction W. The rearward wing unit 41 comprises a pair of transversely extending wings and the low rigidity region 50 lies between the wings. According to such a structure, while the low rigidity region 50 is readily deformed along the concaveness of the centerline of the wearer's body, an excessive deformation (twisting) can be prevented by disposing the rearward wing unit 41 in which an adhesive agent is applied to each side of the low rigidity region 50.

The low rigidity region 50 may be composed of a concave region in which the main absorber 11 is concaved on the side of the backsheet 13. In such a case, the upper layer unit 20 may be configured to be included in such a concave region.

According to such a structure, since the upper absorber 21 has a thickness, the absorber is included in the concave region, thereby reducing a difference between: a total thickness of the upper layer unit 20 and the main body potion 10 in the region in which the upper layer absorber 21 is not disposed; and a total thickness of the upper layer unit 20 and the main body unit 10 in the region in which the upper layer absorber 21 is disposed. Therefore, when the absorbent article 1 is folded onto the skin contact surface side for the sake of package, winkles hardly occur at the skin contact surface side on which the upper layer absorber 21 is disposed, and a gap is hardly produced.

An adhesive agent for bonding the absorbent article 1 with underwear is applied to the underwear contact face side of the backsheet 13.

For example, as shown in FIG. 2(a), an adhesive agent 61 for bonding the absorbent article 1 with underwear is applied to the cloth contact face side of the central wing unit 40, and as shown in FIG. 2(b), an adhesive agent 62 for bonding the absorbent article 1 with underwear is applied to the cloth contact face side of the rear wing unit 41.

In addition, as shown in FIG. 2(b), an adhesive agent 63 for bonding the absorbent article 1 with underwear along the longitudinal direction L is applied to the cloth contact face side of the backsheet 12. The adhesive agent 63 is not applied to a region of the backsheet 13 that is opposite to a region including the center of the widthwise direction W of the low rigidity region 50.

According to such a structure, since the low rigidity region 50 is not fixed to underwear, the region is readily deformed in a convex shape at a pressure which is more than a wearing pressure of underwear by means of a force of the wearer's crotch.

In addition, as shown in FIG. 1, in the absorbent article 1 according to the embodiment, compression grooves 14A, 14B, 14C, and 14D which are configured to compress members from the topsheet 10 to the absorber 13 are provided on the skin contact surface side of the main body unit 10.

Specifically, at least, the part of the compression grooves 14B and 14C that extend in the longitudinal direction L are provided outside of the widthwise direction W more than in the low frigidity region 50.

According to such a structure, in the widthwise direction W, the low rigidity region 50 is sandwiched in a high rigidity region (between the compression grooves 14B and 14C), and is readily deformed so as to be taken along the concave shape of the wearer's body. Further, since the low rigidity region 50 is a region which is readily twisted, twisting durability can be enhanced by disposing the compression grooves 14B and 14C.

Further, a compression groove 14D which extends in the widthwise direction W is provided rear of the longitudinal direction L more than in the low rigidity region 50.

According to such a structure, since a convex-shaped deformation of the low rigidity region 50 hardly propagates up to a rear end of the absorbent article 1, an excessive deformation (twisting) can be prevented. Namely, a rear end portion at which the upper layer absorber 21 is not disposed is configured to come into contact with the wearer's body in a curved shape.

The main body unit 10 and the upper layer unit 20 are bonded with each other by means of the compression grooves 14A and 14D. Specifically, the upper layer topsheet 22, the topsheet 12, and the main absorber 11 are bonded with each other.

In addition, in a region in which the central wing unit 40 is provided, in the widthwise direction W of the absorbent article 1, both edge parts of the upper layer unit 20 are disposed so as not to extend to the outside of the compression groove 14A.

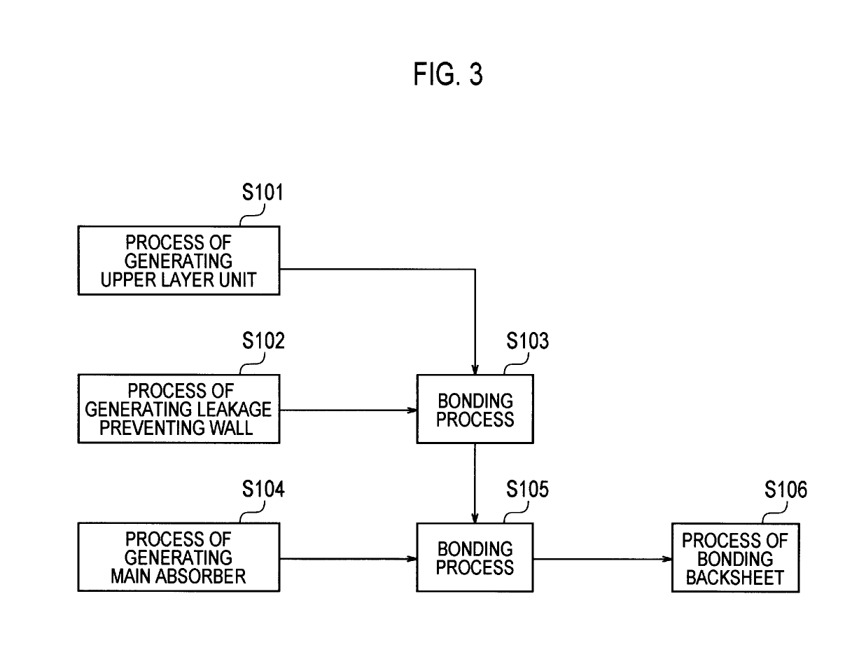

Next, with reference to FIG. 3, a part of a method of manufacturing the absorbent article 1 according to the embodiment will be described. An existing method can be employed as to those which are not described in FIG. 3.

As shown in FIG. 3, in step S101, an upper layer unit 20 is generated.

Specifically, first, a bulk recovery treatment or a cooling treatment is applied to a web for the upper layer topsheet 22 to thereby recover a bulk.

Secondly, the upper layer absorber 21 is cut in a predetermined length; a hot melt adhesive agent is applied to the absorber; and thereafter, the cut absorber is adhered onto the web for the upper layer topsheet 22.

Third, a hot melt adhesive agent is applied onto the web for the upper layer topsheet 22 and the upper layer absorber 21, and then, the web and absorber each are folded into three sections in a sailor process; and the upper layer absorber 21 is lapped by means of the web for the upper layer topsheet 22.

Fourthly, in the web for the supper layer topsheet 22, the abovementioned spacing region is formed by performing emboss processing.

In step S102, a leakage preventing wall 30 is generated.

Specifically, first, a hot melt adhesive agent is applied to an elastic member 32; the elastic member is caused to join a leakage proof sheet 31 which has been partially cut by means of a slitter; and a foundation of the leakage preventing wall 30 is formed through the folding process.

Secondly, the foundation of the leakage preventing wall 30 is caused to join the web for the topsheet 12, and then, by means of emboss processing the foundation of the leakage preventing wall 30 and the web for the topsheet 12 are bonded with each other.

Thirdly, by means of the folding process, a Q-shaped leakage preventing wall 30 is formed from the foundation of the leakage preventing wall 30.

In step S103, a hot melt adhesive agent is intermittently applied to the web for the upper layer topsheet 22 that has been folded into three sections; the web is adhered to a web for the top sheet 12 on which the leakage preventing wall 30 has been formed; and thereafter, by means of emboss processing, the web for the supper layer topsheet 22 and the web for the topsheet 12 are bonded with each other.

In step 104, a main absorber 11 is generated.

Specifically, first, pulp and absorptive polymer crushed on tissue are laminated and then are wrapped by means of the tissue in the folding process, and a foundation of the main absorber 11 is formed.

Secondly, by means of emboss processing a thickness of the foundation of the main absorber 11 is formed to be a predetermined thickness.

Thirdly, by employing an absorber cutter, the foundation of the main absorber 11 is cut into a predetermined dimension and shape, whereby the main absorber 11 is generated.

In step 105, the main absorber 11 is adhered to the web for the topsheet 12 with which the web for the topsheet 22 and the leakage preventing wall 30 are bonded; and on the skin contact surface side of the absorbent article 1, a compression groove of a predetermined shape is formed by means of emboss pressing.

In step S106, a hot melt adhesive agent is applied to a web for a backsheet 13, and then, the web is caused to join the non-skin contact surface side of the absorbent article 1 of the web for the topsheet 12.

(Exemplary Modification 1)

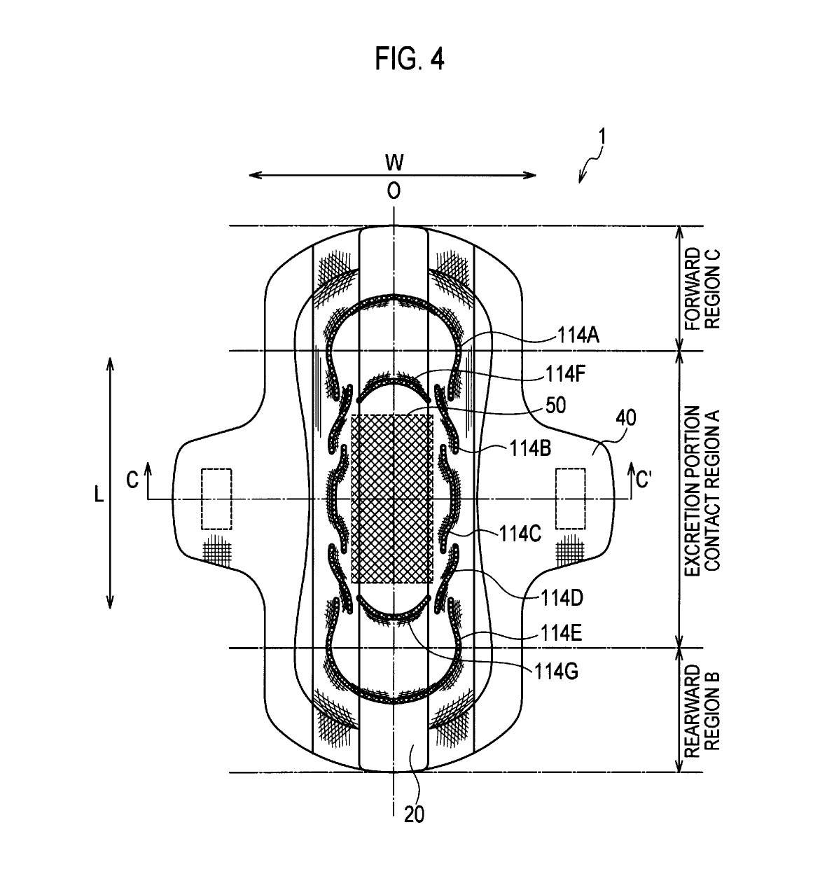

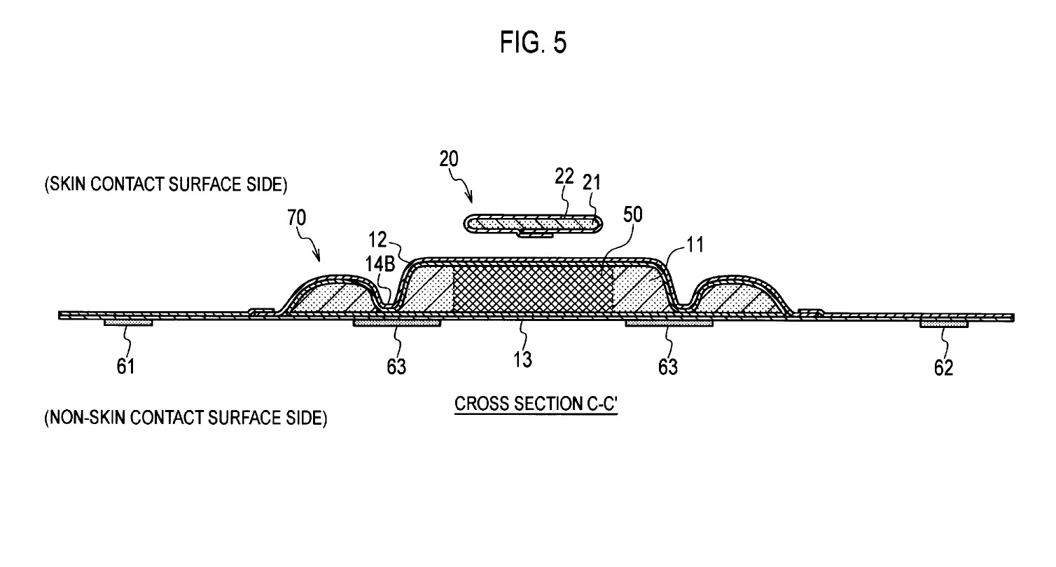

With reference to FIG. 4 and FIG. 5, an absorbent article 1 according to Exemplary Modification 1 of the present invention will be described focusing on differences from the abovementioned absorbent article 1 according to the first embodiment.

As shown in FIG. 4 and FIG. 5, the absorbent article according to Exemplary Modification 1 will be described by way of example of sanitary napkin for daytime. That is, while, in the absorbent article 1 according to Exemplary Modification 1, a central wing unit 40 is provided, a rearward wing unit is not provided.

Here, as shown in FIG. 4, the central wing unit 40 is provided outside of the low rigidity region 50 in the widthwise direction W and the low rigidity region lies between the wings of the central wing unit 40.

In addition, a compression grove 114F which extends in the widthwise direction W is provided forward of the low rigidity region 50 in the longitudinal direction L, and a compression groove 114G which extends in the widthwise direction W is provided rearward of the low rigidity region 50 in the longitudinal direction L.

While the present invention has been described in detail by employing the foregoing embodiment, it is apparent that the present invention is not limitative to the embodiment described in the specification. The present invention can be carried out as aspects of modification and alternation without deviating from the gist and scope of the present invention, defined by the recitations of the claims. Therefore, the description of the present specification is intended for illustrative explanation, and does not have any limitative meaning to the present invention.

For example, the present invention may be described as follows. The first aspect of the present invention described above may be arranged in at least following items:

An absorbent article having: a main body unit comprising a topsheet; a backsheet; and a main absorber which is disposed between the topsheet and the backsheet, wherein: an upper layer unit which extends in a longitudinal direction of the absorbent article is provided on a skin contact surface side of the main body unit; the upper layer unit has an upper layer topsheet which is independent of the topsheet; a dimension of the upper layer unit in a widthwise direction of the absorbent article is configured to be smaller than a dimension of the main body unit in the widthwise direction; a central region is provided in the main absorber, which includes a widthwise center of the main absorber that is opposite to the upper layer unit; and in at least a part of the central region, a low rigidity region is formed which is arranged to have a lower flexural rigidity than that of the central region.

The second aspect of the present invention described above may be arranged in at least following items:

A sanitary napkin including a main body unit having a topsheet, a backsheet, and a main absorber which is disposed between the topsheet and the backsheet, wherein: the main body unit has a central wing unit which extends outwardly in a widthwise direction from opposed longitudinally extending side edges of the sanitary napkin and a rearward wing unit which is provided rearward of the central wing unit in a longitudinal direction of the sanitary napkin and which extends outwardly in the widthwise direction from opposed longitudinally extending side edges of the sanitary napkin; an upper layer unit extending in the longitudinal direction of the sanitary napkin is provided at a skin contact surface side of the main body unit;

the upper layer unit has an upper layer topsheet which is independent of the topsheet and an upper layer absorber which is independent of the main absorber; a dimension of the upper layer unit in the widthwise direction of the sanitary napkin is configured to be smaller than a dimension of the main body unit in the widthwise direction; a central region is provided in the main absorber, which includes a widthwise center of the main absorber that is opposite to the upper layer unit; in the central region, in a region at least a part of which is opposite to a region in which the upper layer absorber is disposed in the upper layer unit, a low rigidity region is formed which is arranged to have a lower flexural rigidity than that of the central region;

the low rigidity region is formed rearward in the longitudinal direction of a region in which a dimension of the main body unit in the widthwise direction is the smallest; the rearward wing unit is provided outside of the low rigidity region in the widthwise direction; the main body unit and the upper layer unit are not bonded with each other in a region which is opposite to the low rigidity region; a dimension of the low rigidity region in the widthwise direction is configured to be smaller than the dimension of the upper layer absorber in the widthwise direction; at least a first compression groove and a second compression groove which are separated from each other are provided on the skin contact surface side of the main body unit; the first compression groove extends in the longitudinal direction outside of the low rigidity region in the widthwise direction; the second compression groove extends in the longitudinal direction outside of the low rigidity region in the widthwise direction and extends in the widthwise direction rearward in the longitudinal direction of a region which is opposite to the low rigidity region; and a basis weight of the main absorber in the low rigidity region is configured to be lower than a basis weight of the main absorber in a peripheral region of the low rigidity region.

1. An absorbent article having: a main body unit including a topsheet; a backsheet; and a main absorber unit which is disposed between the topsheet and the backsheet, wherein:
an upper layer unit which extends in a longitudinal direction of the absorbent article is provided on a skin contact surface side of the main body unit;
the upper layer unit has an upper layer topsheet which is independent of the topsheet;
a dimension of the upper layer unit in a widthwise direction of the absorbent article is configured to be smaller than a dimension of the main body unit in the widthwise direction;
a central region including a center of the widthwise direction of the main absorber that is opposite to the upper layer unit is provided; and in at least a part of the central region, a low rigidity region of which flexural rigidity is lower than that of the central region is formed.

2. The absorbent article according to claim 1, wherein the upper layer unit is not bonded with the main body unit in at least a part of a region which is opposite to the low rigidity region.

3. The absorbent article according to claim 1, wherein:
the upper layer unit has an upper layer absorber which is independent of the main absorber; and
the low rigidity region is formed in a region of the main absorber of which at least a part is opposite to a region in which the upper layer absorber is disposed in the upper layer unit.

4. The absorbent article according to claim 3, wherein
a dimension of the low rigidity region in the widthwise direction is configured to be smaller than a dimension of the upper layer absorber in the widthwise direction.

5. The absorbent article according to claim 1, wherein
a pair of compression groove extending in the longitudinal direction are provided outside of the widthwise direction more than in the low rigidity region, at a skin contact surface side of the main body unit.

6. The absorbent article according to claim 1 wherein
a compression groove extending in the widthwise direction is provided rearward of the longitudinal direction more than in the low rigidity region, at the skin contact surface side of the main body unit.

7. The absorbent article according to claim 1, wherein
a basis weight of the main absorber in the low rigidity region is configured to be lower than a basis weight of the main absorber in a peripheral region of the low rigidity region.

8. The absorbent article according to claim 1, wherein:
the main body unit has a central wing unit which extends to both outsides in the widthwise direction; and
the low rigidity region is formed rearward of the longitudinal direction more than at the central wing unit.

9. The absorbent article according to claim 1, wherein:
an adhesive agent for bonding the absorbent article with an underwear is applied to an underwear contact surface side of the backsheet; and the adhesive agent is not applied to a region of the backsheet that is opposite to a region including a center of the widthwise direction of the low rigidity region.

10. The absorbent article according to claim 1 wherein:
the low rigidity region is comprised of a concave region in which the main absorber is concaved on the backsheet side; and
the upper layer unit is configured to be included in the concave region.

11. The absorbent article according to claim 1, wherein
a central wing unit or a rearward wing unit is provided outside of the widthwise direction of the low rigidity region.

12. A sanitary napkin including a main body unit having a topsheet, a backsheet, and a main absorber which is disposed between the topsheet and the backsheet, wherein:
the main body unit has a central wing unit which extends outwardly in a widthwise direction from opposed longitudinally extending side edges of the sanitary napkin and a rearward wing unit which is provided rearward of the central wing unit in a longitudinal direction of the sanitary napkin and which extends outwardly in the widthwise direction from opposed longitudinally extending side edges of the sanitary napkin;
an upper layer unit extending in the longitudinal direction of the sanitary napkin is provided at a skin contact surface side of the main body unit;
the upper layer unit has an upper layer topsheet which is independent of the topsheet and an upper layer absorber which is independent of the main absorber;
a dimension of the upper layer unit in the widthwise direction of the sanitary napkin is configured to be smaller than a dimension of the main body unit in the widthwise direction;
a central region is provided in the main absorber, which includes a widthwise center of the main absorber that is opposite to the upper layer unit;
in the central region, in a regions at least a part of which is opposite to a region in which the upper layer absorber is disposed in the upper layer unit, a low rigidity region is formed which is arranged to have a lower flexural rigidity than that of the central region;
the low rigidity region is formed rearward in the longitudinal direction of a region in which a dimension of the main body unit in the widthwise direction is the smallest;
the rearward wing unit is provided outside of the low rigidity region in the widthwise direction;
the main body unit and the upper layer unit are not bonded with each other in a region which is opposite to the low rigidity region;
a dimension of the low rigidity region in the widthwise direction is configured to be smaller than the dimension of the upper layer absorber in the widthwise direction;
at least a first compression groove and a second compression groove which are separated from each other are provided on the skin contact surface side of the main body unit;
the first compression groove extends in the longitudinal direction outside of the low rigidity region in the widthwise direction;
the second compression groove extends in the longitudinal direction outside of the low rigidity region in the widthwise direction and extends in the widthwise direction rearward in the longitudinal direction of a region which is opposite to the low rigidity region; and
a basis weight of the main absorber in the low rigidity region is configured to be lower than a basis weight of the main absorber in a peripheral region of the low rigidity region.
